(19) United States
(12) Patent Application Publication
(10) Pub. No.: US 2026/0315375 A1
(43) Pub. Date: Oct. 15, 2026

(54) CANNABIDIOL FORMULATION AND PREPARATION METHODS

(71) Applicant: Qualthera Pharmaceuticals Ltd. Co., Dallas, TX (US)

(72) Inventor: Donald Prentiss, Dallas, TX (US)

(21) Appl. No.: 19/636,516

(22) Filed: Apr. 1, 2026

Related U.S. Application Data
(60) Provisional application No. 63/785,961, filed on Apr. 9, 2025.

(51) Int. Cl. A61K 9/00; A61K 9/16; A61K 31/00

(52) U.S. Cl. CPC A61K 9/0024; A61K 9/1617; A61K 9/1635; A61K 9/1647; A61K 9/1694; A61K 31/658

(57) ABSTRACT

A formulation including a phytocannabinoid is disclosed. The formulation may be compressible into a pellet suitable for subcutaneous implantation and configured to provide sustained release of the phytocannabinoid upon implantation. The formulation may be further configured to fully biodegrade following implantation. The formulation may generally include one or more cannabidiols, structural excipients, and release-modifying excipients. The formulation may include a quantity of structural excipient sufficient to give the pellet sufficient structural integrity for subcutaneous implantation. The formulation may include a quantity of release-modifying excipient sufficient to provide gradual and sustained release of the cannabidiol upon implantation. The formulation may include a therapeutic amount of cannabidiol.

### CROSS REFERENCE TO RELATED APPLICATIONS

This application relates and claims priority to U.S. Provisional Application No. 63/785,961, filed Apr. 9, 2025, and entitled “Cannabidiol Subcutaneous Sustained-Release Biodegradable Implant and Methods of Use,” which is hereby incorporated by reference in its entirety.

### TECHNICAL FIELD

The described examples relate generally to a cannabidiol formulation and preparation methods of the same.

### BACKGROUND

Subcutaneous implants have been employed in FDA-approved applications to deliver hormones and other Active Pharmaceutical Ingredients (“APIs”) over extended durations. For instance, testosterone has been administered using compressed pellet implants that provide steady-state hormone levels for several months. These systems rely on solid-state formulations designed to gradually erode and release the active agent in a controlled manner, without requiring frequent administration or producing significant fluctuations in plasma concentration.

Administration of APIs via subcutaneous implants provides several advantages over typical administration techniques (e.g., oral, topical, injection, etc.). For example, subcutaneous implants avoid the need for frequent dosing, which improves patient adherence and compliance. Further, the implant can be configured to produce a controlled, gradual release of the API advantageously avoiding high absorption peaks and rapid troughs typical in injections or the variable bioavailability issues of oral and topical administration. Notwithstanding, subcutaneous implants may not be fully biodegradable and thus may remain within the patient's tissue for an extended period of time. Furthermore, formulations for subcutaneous implantation are often confronted with technical problems that result in inadequate performance upon implantation. For example, formulations may encounter chemical instability due to, for example, moisture accelerating degradation; formulations may lack the necessary physical stability necessary for subcutaneous implantation that provides meaningful extended release; formulations may present excipient incompatibility causing altered release profiles and API destabilization. Moreover, such formulations may be confronted with preparation problems that inhibit performance at the preparation stage. For example, formulations may encounter content constituent segregation during mixing resulting in non-uniform pellets; formulations may encounter mechanical integrity and cracking during compression or chipping during handling; formulations may exhibit unfavorable surface effects including moisture uptake and oxidation causing unintended release.

Additionally, many APIs are not currently available in formulations suitable for subcutaneous implantation or are imperfectly delivered through such implantation. For example, cannabidiol (“CBD”) is a phytocannabinoid known for its various therapeutic properties and taken by many worldwide to alleviate pain, anxiety, movement disorders, and many other conditions. CBD is typically taken orally, which is accompanied by the aforementioned drawbacks. As such, there remains a need for a CBD formulation suitable for subcutaneous implantation that addresses the aforementioned technical problems in the prior art.

### SUMMARY

In one example, a sustained-release formulation is disclosed. The sustained-release formulation includes a solid matrix configured for subcutaneous implantation. The solid matrix includes a quantity of a phytocannabinoid. The solid matrix further includes at least one structural excipient. The solid matrix further includes at least one release-modifying excipient. The at least one structural excipient is configured to provide structural integrity and controlled dissolution to the solid matrix. The at least one release-modifying excipient is configured to modulate diffusion of the phytocannabinoid from the solid matrix following subcutaneous implantation. The solid matrix is configured to release the quantity of phytocannabinoid and biodegrade during the release.

In another example, the solid matrix includes a compressed pellet.

In another example, the phytocannabinoid includes cannabidiol.

In another example, the at least one release-modifying excipient includes stearic acid and/or magnesium stearate.

In another example, the at least one structural excipient includes polycaprolactone, poly(lactic-co-glycolic acid), polylactic acid, and/or polyvinylpyrrolidone.

In another example, the compressed pellet includes about 5-80 wt % of the cannabidiol, about 5-80 wt % of the at least one structural excipient, and about 5-50 wt % of the at least one release-modifying excipient.

In another example, the quantity of the phytocannabinoid released is between 5% and 50% within 30 days following subcutaneous implantation.

In another example, the quantity of the phytocannabinoid released is between 95% and 100% within 2 to 6 months following subcutaneous implantation.

In another example, the quantity of the phytocannabinoid released is between 5% and 80% within 2 weeks to 3 months following subcutaneous implantation.

In yet another example, the quantity of the phytocannabinoid is configured to treat chronic pain, anxiety, post-traumatic stress disorder, opioid withdrawal, Alzheimer's Disease, Parkinson's Disease, Multiple Sclerosis, and/or neurodegenerative disorders.

In one example, a subcutaneous biodegradable implant is disclosed. The subcutaneous biodegradable implant includes a compressed pellet including a quantity of cannabidiol and a quantity of one or more excipients. The compressed pellet is configured to be subcutaneously implanted. The compressed pellet is configured to provide sustained and prolonged release of the quantity of cannabidiol following subcutaneous implantation. The compressed pellet is configured to provide full drug absorption of the quantity of cannabidiol. The compressed pellet is fully biodegradable.

In another example, the compressed pellet is configured to be implanted within a hypodermis tissue layer.

In another example, the compressed pellet is configured to be implantable via a trocar.

In another example, the compressed pellet is configured to provide sustained and prolonged release of the quantity of cannabidiol over a period of about 2 months to about 6 months.

In another example, the one or more excipients includes at least one structural excipient and at least one release-modifying excipient.

In another example, the one or more excipients includes at least one polymer configured to provide structural integrity to the compressed pellet and at least one release-modifying agent configured to modulate diffusion of the quantity of cannabidiol from the compressed pellet.

In yet another example, the at least one polymer includes polycaprolactone, poly(lactic-co-glycolic acid), polylactic acid, or polyvinylpyrrolidone and the at least one release-modifying agent includes stearic acid or magnesium stearate.

In one example, a method of administering a cannabidiol is disclosed. The method includes implanting, subcutaneously, a compressed pellet. The compressed pellet includes a quantity of a cannabidiol and at least one excipient. The method further includes administering the cannabidiol through sustained release of the quantity of cannabidiol from the compressed pellet. The sustained release comprises full drug absorption of the quantity of cannabidiol over an absorption time. The compressed pellet is configured to fully biodegrade during the sustained release.

In another example, the sustained release extends over a period of time between 2 weeks and 6 months.

In another example, the implanting includes insertion via a trocar.

In another example, the method further includes treating chronic pain, anxiety, post-traumatic stress disorder, opioid withdrawal, Alzheimer's Disease, Parkinson's Disease, Multiple Sclerosis, and/or neurodegenerative disorders via the sustained release of the quantity of cannabidiol.

In yet another example, the at least one excipient includes polycaprolactone, poly(lactic-co-glycolic acid), polylactic acid, polyvinylpyrrolidone, stearic acid, and/or magnesium stearate.

In one example, a formulation is disclosed. The formulation includes a matrix. The matrix includes from 5 to 80 wt % of a phytocannabinoid. The matrix further includes from 5 to 80wt % of a structural excipient. The matrix further includes from 5 to 50 wt % of a release-modifying excipient. The matrix is compressible into a pellet.

In another example, the phytocannabinoid includes cannabidiol.

In another example, the structural excipient is selected from the group consisting of polycaprolactone, poly(lactic-co-glycolic acid), polylactic acid, polyvinylpyrrolidone, and mixtures thereof.

In another example, the release-modifying excipient is selected from the group consisting of includes stearic acid, magnesium stearate, and mixtures thereof.

In another example, the cannabidiol is plant-derived or synthetically produced.

In another example, the structural excipient provides structural integrity to the formulation enabling tablebility.

In yet another example, the release-modifying excipient is configured to bind to the phytocannabinoid and control its release from the matrix.

In one example, a solid matrix is disclosed. The solid matrix includes the formulation described in paragraph [0028].

In another example, the solid matrix includes a compressed pellet.

In another example, the pellet is a 100 mg pellet having of length of 9 mm and a width of 3 mm.

In yet another example, the solid matrix further includes a plurality of pellets each having the formulation of paragraph [0028].

In one example, a method for preparing a pellet is disclosed. The method includes compressing a matrix of constituents into the pellet. The matrix of constituents includes from 5 to 80 wt % of a phytocannabinoid. The matrix of constituents further includes from 5 to 80 wt % of a structural excipient. The matrix of constituents further includes from 5 to 50 wt % of a release-modifying excipient.

In another example, the phytocannabinoid includes cannabidiol. The structural excipient is selected from the group consisting of polycaprolactone, poly(lactic-co-glycolic acid), polylactic acid, polyvinylpyrrolidone, and mixtures thereof. The release-modifying excipient is selected from the group consisting of includes stearic acid, magnesium stearate, and mixtures thereof.

In another example, the compressing step includes providing a homogenous mixture of the matrix of constituents and compressing the homogeneous mixture into a cylindrical pellet.

In another example, the matrix of constituents are mixed by double-cone blender, ribbon blender, or V-blender.

In another example, the homogenous mixture is compressed by stamping press rotary or rotary press.

In yet another example, the pellet is a 100 mg pellet having of length of 9 mm and a width of 3 mm.

### BRIEF DESCRIPTION OF THE DRAWINGS

FIG. 1 illustrates a graph showing an example release profile of cannabidiol.

FIG. 2 illustrates an example compressed pellet.

FIG. 3 illustrates an example subcutaneous implantation.

FIG. 4A illustrates the example solid matrix implant of FIG. 2 in an undegraded state.

FIG. 4B illustrates the example solid matrix implant of FIG. 2 in a partially degraded state.

FIG. 4C illustrates the example solid matrix implant of FIG. 2 in a completely degraded state.

FIG. 5 illustrates a flow diagram of an example method for administering a cannabidiol.

FIG. 6 illustrates a flow diagram of an example method for preparing a pellet.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

The description that follows includes example systems, methods, and designs that embody various embodiments of the present invention. However, it should be understood that the described embodiments may be practiced in a variety of forms in addition to those described herein.

As used herein, the term “active pharmaceutical ingredient” or the abbreviation “API” refers to any biologically active ingredient used or included in a drug formulation. In this regard, a substance may be labeled as an API where it is intended to be the primary active ingredient included in the formulation to produce the desired effect (e.g., treatment of a medical condition, ailment, or the like).

As used herein, the term “phytocannabinoid” refers to several structural classes of compounds found in the cannabis plant and/or synthetic compounds mirroring the same. For example, tetrahydrocannabinol (THC) and cannabidiol (CBD) may be labeled as types of phytocannabinoid.

As used herein, the term “subcutaneous implantation” and “subcutaneous implant” refers to the act of and the substance intended to be injected into human tissue, particularly the layer of skin below the dermis and epidermis. For example, a substance injected into the hypodermis layer of a person's skin may be labeled as being subcutaneously implanted. As such, the “subcutaneous layer” or “subcutaneous tissue layer” refers to tissue layers under the skin.

As used herein, the term “treating” and derivatives including “treat,” “treatment,” etc. generally refer to reversing, alleviating, inhibiting the progress of, preventing a disorder or condition in a patient, preventing one or more symptoms of such disorder or condition, and/or otherwise mitigating or reducing (even minimally) the side effects of a condition, ailment, disorder, or the like.

The following disclosure relates generally to a formulation of a solid matrix including a cannabidiol. In several embodiments and as described in detail herein, the formulation is designed for and may be suitable for subcutaneous implantation into a human patient. The formulation of the present disclosure may exhibit a sustained-release and controlled dissolution of the CBD constituent and complete bio absorption upon subcutaneous implantation. Additionally, the following disclosure relates method of preparing the formulation including cannabidiol In several embodiments, the formulation includes a phytocannabinoid (e.g., CBD), a structural excipient, and a release-modifying agent. However, the formulation may include additional components. In several embodiments, the formulation is compressible into a pellet suitable for subcutaneous implantation. The formulation, as described herein, may be used to administer a quantity of CBD via implantation into the subcutaneous layer of a person's skin. The formulation of the present disclosure may be in the form of a compressed solid matrix including constituents of the formulation and designed to deliver a therapeutic quantity of CBD over an extended period of time through controlled dissolution of the solid matrix following implantation.

CBD, and phytocannabinoid compounds in general, are known to impart beneficial health effects and are commonly used to alleviate several aliments. Further, CBD may be considered an attractive medical remediation alternative for those desiring a naturalist approach to medical treatment. As such, CBD has become a widely used and acceptable form of medical treatment. CBD is typically administered orally or topically. However, as will be understood by those having skill in the art, oral or topical administration of APIs (e.g., CBD) present several drawbacks and disadvantages. For example, oral administration of CBD may necessitate consistent and thorough patient adherence. In this regard, a patient may be instructed to take CBD multiple times a week or even a day, which can prove to be a cumbersome task for many. Further, oral administration may exhibit poor pharmacokinetics (e.g., sharp peak and trough drug absorption, variable systemic exposure, etc.), erratic absorption patterns, and poor bioavailability (e.g., reduced absorption from first-pass metabolism).

Additionally, formulations designed for subcutaneous implantation may be confronted with technical problems that result in inadequate performance upon implantation. For example, formulations may encounter chemical instability due to, for example, moisture accelerating degradation; formulations may lack the necessary physical stability necessary for subcutaneous implantation that provides meaningful extended release; formulations may present excipient incompatibility causing altered release profiles and API destabilization. Moreover, such formulations may be confronted with preparation problems that inhibit performance at the preparation stage. For example, formulations may encounter content constituent segregation during mixing resulting in non-uniform pellets; formulations may encounter mechanical integrity and cracking during compression or chipping during handling; formulations may exhibit unfavorable surface effects including moisture uptake and oxidation causing unintended release.

To mitigate these and other challenges, disclosed herein is a formulation including CBD designed for subcutaneous implantation into a human patient. In several embodiments, the formulation includes a matrix of phytocannabinoid, structural excipient, and a release-modifying excipient. The matrix may be compressible into a pellet suitable for subcutaneous implantation. Upon implantation, the formulation may be configured to impart sustained-release of a therapeutic quantity of CBD, thereby gradually releasing the quantity of CBD and causing full dissolution of the pellet such that the pellet is completely degraded and absorbed. Additionally, a method of preparing the pellet including the formulation matrix is described herein.

In several embodiments, the formulation includes a quantity of CBD, a quantity of structural excipient, and a quantity of release-modifying excipient (sometimes collectively referred to as “constituents” herein). The aforementioned quantities may be represented as wight percents (“wt %”) of a total formulation weight. The formulation may be configured to compressed into a pellet suitable for implantation into a subcutaneous tissue layer. The formulation may be designed to impart sustained and extended release of the CBD and full biodegradation within the patient's tissue during drug delivery following implantation. The formulation of constituents may be in certain proportions to effectuate sustained release of the cannabidiol and complete degradation of the solid matrix within a subcutaneous tissue layer. Advantageously, by providing such a sustained and extended release of CBD via subcutaneous implantation the drawbacks in the prior art can be overcome. Additionally or alternatively, the formulation may be designed to be compressible into a solid matrix that overcomes the drawbacks present in the prior art.

In one example, sustained, gradual release of the CBD following implantation includes administration of a 6-month therapeutic quantity of CBD via a single implantation. Advantageous, this avoids the patient having to regularly one or more tablets a day.

In several embodiments, the formulation includes a quantity of one or more structural excipients. In several embodiments, the structural excipients are a polymer-based excipient configured to provide structural stability to the formulation for compressibility into a pellet. In this regard, the structural excipients may provide tabletability to the solid matrix so that it may be compressed into a pellet. In one embodiment, the structural excipient may provide structure integrity to enable compression of constituents into the solid matrix (e.g., compressed pellet, tablet, pill, capsule, caplet, etc.) at a given compression pressure (e.g., via vertical press). In one embodiment, the structural excipient is configured to bind and lubricate the quantity of CBD during preparation of the solid matrix. In one example, the structural excipient provides requisite structural integrity to the solid matrix to enable subcutaneous implantation, that is to ensure the solid matrix remains whole during manufacture, transportation, handling, and said implantation. In several embodiments, the structural excipient is polycaprolactone, poly(lactic-co-glycolic acid), polylactic acid, polyvinylpyrrolidone, or mixtures thereof. However, other structural excipients known by those having skill in the art may be included in the formulation. In several embodiments, the structural excipient is included in the formulation in a quantity representable as a wt % based on total formulation weight. For example, the structural excipient may be 5 to 80 wt % of the formulation. Advantageously, the structural excipient may enable the formulation to be compressed in the solid matrix (e.g., pellet) and provide structure integrity for subcutaneous implantation. In one embodiment, the structural excipient further provides controlled dissolution of the constituents following implantation.

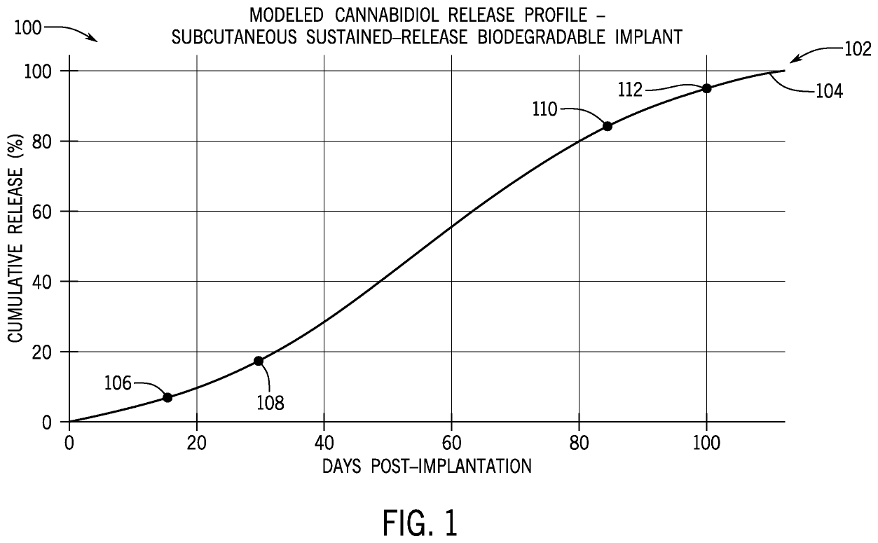

In several embodiments, the formulation includes a quantity of one or more release-modifying excipients. In several embodiments, the release-modifying excipients are configured to control the release of CBD from the solid matrix following implantation. In several embodiments, the release-modifying excipient is configured to bind to the CBD and gradually unbind following implantation to effectuate the sustained release of the CBD into the patient. In this regard, controlled release may cause a smooth, plateau-like drug release (e.g., as illustrated in FIG. 1) for consistent therapeutic levels while avoiding a high initial peak. For example, the release-modifying excipient may enable an extended release of the CBD (e.g., up to 6 months of continually drug release). In several embodiments, the release-modifying excipient is stearic acid, magnesium stearate, or mixtures thereof. However, other release-modifying excipients known by those having skill in the art may be included in the formulation. In several embodiments, the release-modifying excipient is included in the formulation in a quantity representable as a wt % based on total formulation weight. For example, the release-modifying excipient may be 5 to 50% wt % of the formulation. Advantageously, the release-modifying agent may enable gradual release of the CBD from the pellet following subcutaneous implantation.

In one example, the formulation includes a therapeutic quantity of CBD sufficient to treat several conditions and ailments. For example, the quantity of CBD may be a therapeutic quantity configured to treat chronic pain, anxiety, post-traumatic stress disorder, opioid withdrawal, Alzheimer's Disease, Parkinson's Disease, Multiple Sclerosis, and/or other disorders.

The aforementioned constituents (e.g., CBD, structural excipient, and release-modifying excipient) may be accompanied by other compounds not specifically recited herein but understood by those skilled in the art to effectuate drug deliver and controlled dissolution of the subcutaneous implant (e.g., fillers/diluents, binders, disintegrants, lubricants/glidants, coating agents, colorants/flavors, preservatives, etc.).

The formulation of the present disclosure may include a drug formulation including the constituents in particular proportions to produce the desired effects. Such proportions may be altered to produce, for example, prolonged and sustained drug release and dissolution, rapid drug release and dissolution, and balances thereto based on the need. Generally, the proportion of the excipients may be altered to control the timeline of dissolution of the solid matrix. For example, a high proportion of structural excipients may be included to produce a longer dissolution timeline, while a lower proportion of structural excipients may be included to produce a shorter dissolution time. Generally, the proportion of the release-modifying excipient may be altered to modulate drug diffusion of the CBD to the patient from the solid matrix. For example, a high proportion of release-modifying excipients may be included to produce a longer drug diffusion timeline, while a lower proportion of release-modifying excipients may be included to produce a shorter drug diffusion timeline. Generally, the proportion of CBD (or the quantity of CBD) included in the solid matrix may be altered to control the therapeutic effects of the CBD. In this regard, the proportion or quantity of CBD may be proportional to the therapeutic needs of the patient (e.g., a high level of chronic pain requiring a high quantity of CBD) or the tolerance of the patient (e.g., a low tolerance to CBD requiring a low quantity of CBD). Additionally, the drug formulation proportions may be altered in consideration of implantation of more than one CBD subcutaneous sustained-release implant (e.g., 10 100 mg pellets for a 1000 mg dose).

In several embodiments, the formulation includes about 5-80 weight percent of the cannabidiol, about 5-80 weight percent of the at least one structural excipient, and about 5-50 weight percent of the at least one release-modifying excipient.

For example, the amount of CBD within the formulation may be from 5% (e.g., 5 mg CBD in a 100 mg pellet) to 10% (e.g., 10 mg in a 100 mg pellet), from 10% to 15% (e.g., 15 mg in a 100 mg pellet), from 15% to 20% (e.g., 20 mg in a 100 mg pellet), from 20% to 25% (e.g., 25 mg in a 100 mg pellet), from 25% to 30% (e.g., 30 mg in a 100 mg pellet), from 30% to 35% (e.g., 35 mg in a 100 mg pellet), from 35% to 40% (e.g., 40 mg in a 100 mg pellet), from 40% to 45% (e.g., 45 mg in a 100 mg pellet), from 45% to 50% (e.g., 50 mg in a 100 mg pellet), from 50% to 55% (e.g., 55 mg in a 100 mg pellet), from 55% to 60% (e.g., 60 mg in a 100 mg pellet), from 60% to 65% (e.g., 65 mg in a 100 mg pellet), from 65% to 70% (e.g., 70 mg in a 100 mg pellet), from 70% to 75% (e.g., 75 mg in a 100 mg pellet), or from 75% to 80% (e.g., 80 mg in a 100 mg pellet).

For example, the amount of structural excipient within the formulation may be from 5% (e.g., 5 mg structural excipient in a 100 mg pellet) to 10% (e.g., 10 mg in a 100 mg pellet), from 10% to 15% (e.g., 15 mg in a 100 mg pellet), from 15% to 20% (e.g., 20 mg in a 100 mg pellet), 20% to 25% (e.g., 25 mg in a 100 mg pellet), from 25% to 30% (e.g., 30 mg in a 100 mg pellet), from 30% to 35% (e.g., 35 mg in a 100 mg pellet), from 35% to 40% (e.g., 40 mg in a 100 mg pellet), from 40% to 45% (e.g., 45 mg in a 100 mg pellet), from 45% to 50% (e.g., 50 mg in a 100 mg pellet), from 50% to 55% (e.g., 55 mg in a 100 mg pellet), from 55% to 60% (e.g., 60 mg in a 100 mg pellet), from 60% to 65% (e.g., 65 mg in a 100 mg pellet), from 65% to 70% (e.g., 70 mg in a 100 mg pellet), from 70% to 75% (e.g., 75 mg in a 100 mg pellet), or from 75% to 80% (e.g., 80 mg in a 100 mg pellet).

For example, the amount of release-modifying excipient within the formulation may be 5% (e.g., 5 mg release-modifying excipient in a 100 mg pellet) to 10% (e.g., 10 mg in a 100 mg pellet), from 10% to 15% (e.g., 15 mg in a 100 mg pellet), from 15% to 20% (e.g., 20 mg in a 100 mg pellet), from 20% to 25% (e.g., 25 mg in a 100 mg pellet), from 25% to 30% (e.g., 30 mg in a 100 mg pellet), from 30% to 35% (e.g., 35 mg in a 100 mg pellet), from 35% to 40% (e.g., 40 mg in a 100 mg pellet), from 40% to 45% (e.g., 45 mg in a 100 mg pellet), or from 45% to 50% (e.g., 50 mg in a 100 mg pellet).

As discussed, the formulation of the present disclosure may include a matrix of constituents compressible into a solid matrix. In several embodiments, the solid matrix is a pellet suitable for subcutaneous implantation. In several embodiments, the formulation is configured to provide a gradual and sustained release of the CBD over an extended period of time following implantation. Advantageously, by administering the CBD via subcutaneous implantation, the shortcomings and disadvantages accompanying oral administration are avoided. In this regard, the formulation may be configured to release the quantity of CBD over a period of one month, three months, or six months following implantation. However, the formulation may be configured to release the quantity of CBD over a period of time less than one month or more than six months following implantation. In one example, the formulation is designed to cause the quantity of CBD released to be between 5% and 50% of the quantity of CBD within the solid matrix formulation withing 30 days following subcutaneous implantation. In one example, the formulation is designed to cause the quantity of CBD released to be between 95% and 100% within two to six months following subcutaneous implantation. In one example, the formulation is designed to cause the quantity of CBD released to be between 20% and 80% within two weeks to three months following subcutaneous implantation.

FIG. 1 illustrates a graph 100 showing an example release profile 102 the cannabidiol. Graph 100 illustrates an example gradual and extended release profile of cannabidiol in a patient following. In several embodiments, the formulation of the present disclosure may be formulated to exhibit the release profile of graph 100 upon implantation. Graph 100 includes CBD release profile 104 illustrating a release of the quantity of CBD from, for example, a solid matrix of the formulation of the present disclosure following implantation. In one example, CBD release profile 102 is associated with a the release profile of the formulation of constituents including a quantity CBD as disclosed herein. For illustrative purposes, graph 100 plots the cumulative release of the quantity of CBD in the form of a percentage over days following implantation. As will be understood by those having skill in the art, example release profile 102 illustrates merely one example release profile that the formulation of the present disclosure may be design to cause upon implantation and that the release profile may be modified based on the propositions of constituents included in the formulation based on the need. In one example, release profile 102 is configured to treat chronic pain, anxiety, post-traumatic stress disorder, opioid withdraw, Alzheimer's Disease, Parkinson's Disease, Multiple Sclerosis, and other disorders.

The release profile 102 of FIG. 1 illustrates the advantages the subcutaneous implantation has over prior methods of administering CBD (e.g., orally). For example, release profile 102 illustrates a sustained and extended release of a quantity of CBD over a period of 100 to 115 days while only requiring a single implantation (rather than numerous daily oral dosages). Furthermore, and as will be understood by one having skill in the art in light of the present disclosure, the formulation may be designed such that the solid matrix degrades contemporaneously with the release of CBD, such that the solid matrix is completely absorbed by the patient's body following full release and absorption of the quantity of CBD (e.g., after about 100 to 115 days). Additionally or alternatively, the formulation may be configured to produce uniform degradation as the CBD releases.

As illustrated in FIG. 1, release profile 102 may include cumulative release point 106 that includes about 5% of the quantity of CBD of the formulation released from the solid matrix at around 14 days following implantation. Release profile 102 may further include cumulative release point 108 that includes about 18% of the quantity of CBD of the formulation released from the solid matrix at around 30 days following implantation. Release profile 102 may further include cumulative release point 110 that includes about 85% of the quantity of CBD of the formulation released from the solid matrix at around 85 days following implantation. Release profile 102 may include cumulative release point 112 that includes about 95% of the quantity of CBD of the formulation released from the solid matrix at around 100 days following implantation. The aforementioned cumulative release points are included for clarity and one having skill in the art will appreciate that numerous cumulative release points are illustrated in FIG. 1.

In several embodiments, the formulation of the present disclosure includes a matrix of constituents, which may be compressible into a solid matrix, such as a cylindrical pellet. In one embodiment, the pellet is suitable for subcutaneous implantation. In this regard, the pellet may be of a small enough size for subcutaneous implantation. In one example, the solid matrix is a compress pellet about the size of a grain of rice (e.g., about 9 mm in length and 3 mm in width).

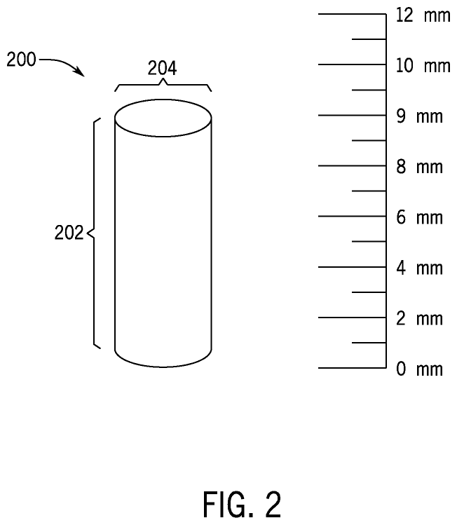

FIG. 2 illustrates an example compressed pellet 200. In several embodiments, compressed pellet 200 includes one or more of the example formulations described herein. In several embodiments, compressed pellet 200 is suitable for subcutaneous implantation. In this regard, compressed pellet 200 may have sufficient structural integrity to remain, at least substantially solid during manufacture, transportation, handling, and implantation; may be of a sufficiently small size to be subcutaneously implanted; and may otherwise be of a shape suitable for implantation via, for example, a trocar.

The example compressed pellet 200 may be the solid matrix described herein and include the formulations as disclosed herein, that is, a formulation including a quantity of CBD, at least one structural excipient, and at least one release-modifying excipient. In this regard, compressed pellet 200 may include the various formulation quantities and proportions described herein. Compressed pellet 200 may be configured for subcutaneous implantation. In this regard, compressed pellet 200 may be of a size and shape for subcutaneous implantation and subsequent drug release thereafter. In one example, compressed pellet 200 includes a length 202 of about 9 mm and a width 204 of about 3 mm. However, the compressed pellet of the present disclosure may have a length and width smaller or larger than that depicted in FIG. 2. In one example, compressed pellet 200 is a generally cylindrical shape.

The formulation of the present disclosure may include several constituents formed into a solid matrix, such as a compressed pellet. In one example, the constituents (e.g., CBD, structural excipient, and release-modifying excipient) are homogenously mixed via specialized equipment, such as a double cone blender, ribbon blender, V-blender, homogenizer, etc. Thereafter, the homogenous mixture of constituents may be compressed into a cylindrical shape via specialized equipment, such as a stamping press, rotary press, etc.

In several embodiments, pellet 200 includes a quantity of structural excipient to provide structure integrity for said subcutaneous implantation such that the pellet does not disintegrate, flake, chip, or otherwise degrade during implantation. The structural excipient may further provide for the controlled dissolution of the solid matrix such that it gradually disintegrates in a controlled fashion following implantation. The pellet 200 may further include a quantity of release-modifying excipient to modulate drug diffusion from the solid matrix following implantation. Subcutaneous implantation may include implanting the compressed pellet into a patient's tissue.

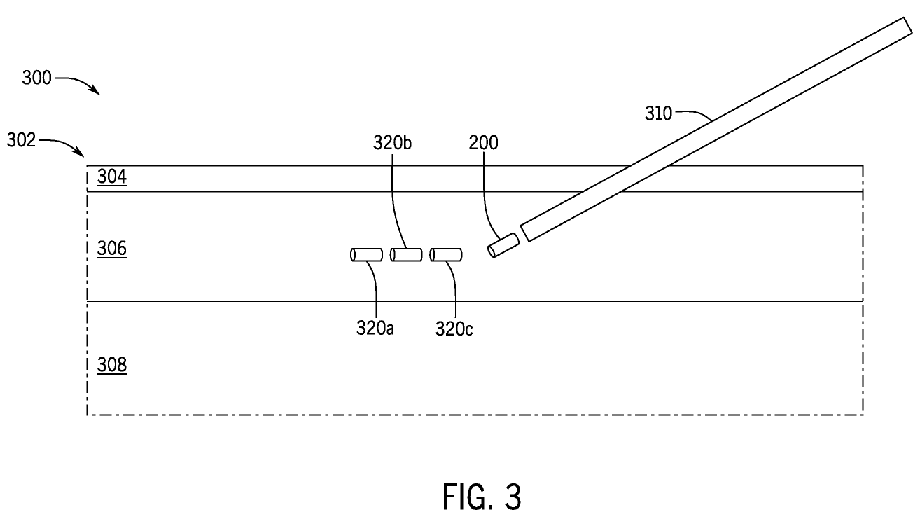

FIG. 3 illustrates an example subcutaneous implantation 300. In one example, subcutaneous implantation 300 includes implanting compressed pellet 200, having the formulation described herein, into a patient's tissue 302. In one example, subcutaneous implantation 300 includes implanting several compressed pellets. Tissue 302 may generally include an upper skin layer 304, a subcutaneous tissue layer 306, and a muscle layer 308. Implantation 300 may be effectuated via use of minimal invasive surgical equipment operable to insert compressed pellet 200 within tissue 302. In one example, compressed pellet 200 is implanted via trocar 310 through the skin layer 304 and into the subcutaneous tissue layer 306. In one example, the subcutaneous tissue layer 304 is a hypodermis layer. In this regard, the compressed pellet 200 may be disposed within the subcutaneous tissue layer 304 for administration of the quantity of CBD contained therein. For example, following implantation a capillary network may be formed around compressed pellet 200 that slowly erodes the compressed pellet 200, absorbing the quantity of CBD contained therein. In one example, disintegration and CBD release of compressed pellet 200 occurs via the example drug release profile 104 of FIG. 1.

Additionally or alternatively, the formulation may be configured and/or proportioned to be one of several pellets suitable for subcutaneous implantation. In this regard, the formulation may be configured such that the complete therapeutic amount of CBD intended to be administered is the additive value of all pellets. Those skilled in the art will appreciate that the formulation and associated example proportions, as represented as wt % based on total formulation weight, may be applicable to more than one pellet.

In one example and as illustrated in FIG. 3, more than one compressed pellet may be implanted to effectuate treatment. For example, compressed pellets 320a, 320b, 320c (which may be substantially analogous to compressed pellet 200) may be implanted in addition to compressed pellet 200. In this regard, treatment may involve administration of a dosage equal to the collective dosage of compressed pellets 200, 320a, 320b, 320c. For example, treatment may require administration of 200 mg of CBD, which can be effectuated by implantation of four compressed pellets (e.g., compressed pellets 200, 320a, 320b, 320c) each including 50 mg of CBD. Advantageously, the compressed pellets described herein need not be increased in size to meet the required treatment dosage and implantation of one or more compressed pellet provides treatment flexibility based on the patient's needs.

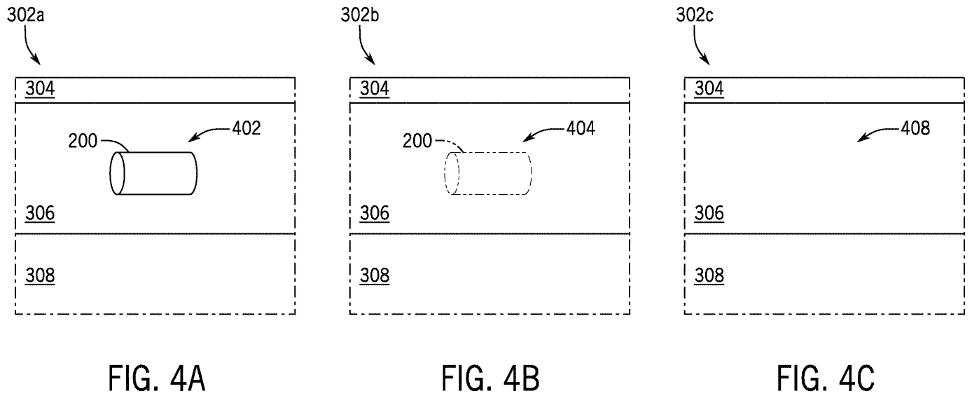

The compressed pellet 200 (and compressed pellets 320a, 320b, 320c) may be configured to completely disintegrate and be fully absorbed by tissue 304 following implantation. In several embodiments, the ability to completely disintegrate and be fully adsorbed is contributable, at least partially, to the example formulations described herein. In this regard, and with reference to FIGS. 4A-4C, the compressed pellet 200 may fully biodegrade within the subcutaneous layer 304 contemporaneous with the quantity of CBD within compressed pellet 200 being fully absorbed by the patient. In one example, full absorption includes 100% of the quantity of CBD being removed from the compressed pellet 200 and absorbed by the patient, thereby effectuating treatment. In one example, full biodegradation includes complete disintegration of the compressed pellet 200, such that no solid matrix remains following the lifecycle of the pellet 200. In one example, and in contrast to the prior art, compressed pellet 200 is configured to degrade uniformly as the CBD is released. In this regard, as opposed to leaving behind a drug depleted matrix following API release that then subsequently biodegrades overtime, the compressed pellet 200 (and subcutaneous implantation of the present disclosure as a whole) does not leave behind a drug depleted matrix but degrades uniformly alongside CBD release. In one example, such drug release and contemporaneous degradation is effectuated by the formulation of CBD, structural excipient, and release-modifying excipient.

Turning specifically to FIG. 4A, which illustrates compressed pellet 200 implanted within a patient's tissue 302 in an undegraded state 402. Undegraded state 402 represents compressed pellet 200 prior to or at least only minimally degraded by absorption of the constituents therein. Upon initial implantation within the subcutaneous tissue layer 306, compressed pellet 200 may be in an undegraded state 402. Undegraded state 402 may represent the compressed pellet 200 with a minimal amount of CBD absorbed by a patient's body and minimal degradation of the compressed pellet 200. For example, undegraded state 402 may include compressed pellet 200 with 0% to 10% CBD absorption. In another example, undegraded state 402 includes compressed pellet 200 with 0% to 25% CBD absorption. In another example, undegraded state 402 includes compressed pellet 200 with 0% to 50% CBD absorption. Undegraded state 402 may be presently shortly after implantation 300, for example within the first hour to two weeks following implantation 300. In another example, undegraded state 402 is present within the first hour to two weeks, three weeks, one month, or longer following implantation based on the quantity of release-modifying agent and excipient included in compressed pellet 200. In this regard, undegraded state 402 illustrates the state of compressed pellet 200 prior to or during initial capillary network construction around compressed pellet 200 by the patient's body.

Turning specifically to FIG. 4B, which illustrates compressed 200 implanted within a patient's tissue 302 in partially degraded state 404. Partially degraded state 404 represents compressed pellet 200 partially or substantially degraded by absorption of the continents therein. Following implantation 300 or at least sometime after implantation 300, within the subcutaneous tissue layer 306, compressed pellet 200 may be in partially degraded state 404. Such partially degraded state 404 may be present following or after a substantial time following capillary network construction around compressed pellet 200 by the patient's body. Partially degraded state 404 may represent the compressed pellet 200 with a partial or substantial amount of CBD absorbed by a patient's body. For example, partially degraded state 404 may include compressed pellet 200 with 10% to 25% CBD absorption. In another example, partially degraded state 404 includes compressed pellet 200 with 10% to 50% CBD absorption. In another example, partially degraded state 404 includes compressed pellet 200 with 10% to 75% CBD absorption. In another example partially degraded state 404 includes compressed pellet 200 with 50% to 99% CBD absorption. Partially degraded state 404 may be presently following implantation 300, for example within the first month to two months following implantation 300. In another example, partially degraded state 404 is present within the first month to 90-days, three months, six months, or longer following implantation based on the quantity of release-modifying agent included in compressed pellet 200.

Turning now specifically to FIG. 4C, which illustrates the lack of compressed pellet 200 within a patient's tissue 302 representative of a completely degraded state 408. Completely degraded state 408 represents how compressed pellet 200 is fully and completely degraded by absorption of the continents therein. For clarity, degraded state 408 does not illustrate a drug depleted matrix that may then slowly degrade following drug release, rather degraded state 408 illustrates how the compressed pellet 200 may be completely absorbed contemporaneous with full CBD drug absorption. Following implantation 300 or at least sometime after implantation 300, compressed pellet 200 may be completely degraded, such that no remnants of compressed pellet 200 are present in the subcutaneous tissue layer 306. In effect, compressed pellet 200 and all constituents thereof is absorbed by the patient's body. Advantageously, the subcutaneous CBD implant of the present disclosure includes a formulation configured for complete biosorption, such the no extraction or shell remnants remain in the patient's tissue 302. Completely degraded state 408 may represent the compressed pellet 200 with all or substantially all the quantity of CBD absorbed by a patient's body. For example, completely degraded state 408 may include compressed pellet 200 with 99% to 100% CBD absorption. Completely degraded state 408 may be presently following implantation 300, for example following the first month from implantation 300. In another example, completely degraded state 408 is present following the second month to three months, four months, five months, six months following implantation, or longer following implantation based on the quantity of release-modifying agent included in compressed pellet 200.

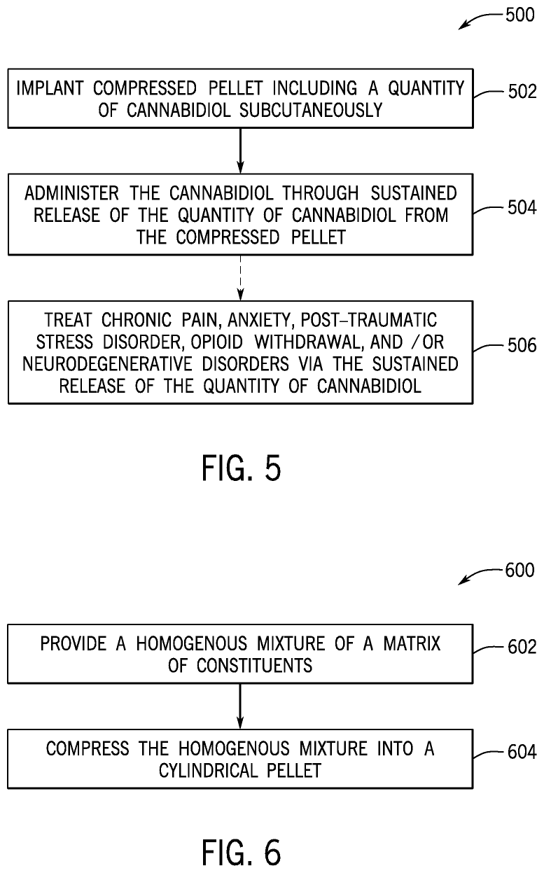

FIG. 5 illustrates a flow diagram 500 of an example method for administering a cannabidiol. At step 502, a compressed pellet including a quantity of CBD is subcutaneously implanted. In one example, the quantity of CBD is of a therapeutic quantity configured to treat several ailments. In one example, the compressed pellet is compressed pellet 200 of FIG. 3 and is implanted via implantation 300 (i.e., utilizing trocar 310) into a subcutaneous tissue layer 306. In this regard, the quantity of CBD within compressed pellet 200 may be accompanied by at least one structural excipient and at least one release-modifying excipient. Compressed pellet 200 may be configured to include the constituent proportions described herein.

At step 504, the CBD is administered through sustained release of the quantity of CBD from the compressed pellet. In one example, sustained release includes a gradual, controlled release of the CBD from the compressed pellet avoiding intense absorbance peaks and troughs. In one example, sustained release of the quantity of CBD occurs in accordance with the CBD release profile 104 illustrated in FIG. 1. In one example, the sustained release of step 504 includes the gradual degradation of compressed pellet 200 as illustrated by FIGS. 4A-4B. In this regard, following sustained release of the quantity of CBD the compressed pellet may be completely degraded and absorbed by a patient's body.

In one example, the sustained release extends over a period of time between 2 weeks to 6 months following implantation. In one example, the full absorption time of the CBD is 6 months following implantation.

In one example, the administering step includes step 506 including treating chronic pain, anxiety, post-traumatic stress disorder, opioid withdrawal, Alzheimer's Disease, Parkinson's Disease, Multiple Sclerosis, and/or neurodegenerative disorders. Such treatment may be effectuated via the sustained release of the quantity of cannabidiol. In one example, treatment is effectuated by the sustained release of the CBD (and other constituents) following implantation. In this regard, the compressed pellet may be configured to gradually release the quantity of CBD into the patient's subcutaneous tissue layer following implantation and while degrading (e.g., as illustrated in FIG. 1). In one example, the rate of release is affected by the quantity and composition of the release-modifying excipient included in the compressed pellet.

The formulation of the present disclosure may be compressed into a pellet. In some embodiments, the pellet is suitable for subcutaneous implantation.

FIG. 6 illustrates a method 600 for preparing a pellet. In several embodies, the pellet includes one or more of the example formulations described herein. At step 602, a homogenous mixture of a matrix of constituents is provided. For example, the matrix of constituents may comprise from 5 to 80 wt % of a phytocannabinoid, 5 to 80 wt % of a structural excipient, and 5 to 50 wt % of a release-modifying excipient. In several embodiments, the phytocannabinoid is cannabidiol. In several embodiments, the structural excipient is selected from the group consisting of polycaprolactone, poly(lactic-co-glycolic acid), polylactic acid, polyvinylpyrrolidone, and mixtures thereof. In several embodiments, the release-modifying excipient is selected from the group consisting of comprises stearic acid, magnesium stearate, and mixtures thereof. In one embodiment, the matrix of constituents is mixed into a homogenous mixture via, for example, double cone blender, ribbon blender, V-blender, or homogenizer.

At step 604 the homogenous mixture is compressed into a cylindrical pellet. In several embodiments, the cylindrical pellet is substantially analogous to pellet 200. In several embodiments, the cylindrical pellet is suitable for subcutaneous implantation (e.g., having a sufficient size, shape, and structural integrity). In several embodiments, the pellet is a 100 mg pellet having a length of 9 mm and a width of 3 mm. In several embodiments, the homogenous mixture is compressed by a stamping press rotary or rotary press.

Other examples and implementations are within the scope and spirit of the disclosure and appended claims. For example, features implementing functions may also be physically located at various positions, including being distributed such that portions of functions are implemented at different physical locations. The foregoing description, for purposes of explanation, uses specific nomenclature to provide a thorough understanding of the described examples. However, it will be apparent to one skilled in the art that the specific details are not required in order to practice the described examples. Thus, the foregoing descriptions of the specific examples described herein are presented for purposes of illustration and description. They are not targeted to be exhaustive or to limit the examples to the precise forms disclosed. It will be apparent to one of ordinary skill in the art that many modifications and variations are possible in view of the above teachings.

What is claimed is:

1. A formulation comprising:
a matrix comprising
from 5 to 80 wt % of a phytocannabinoid;
from 5 to 80 wt % of a structural excipient; and
from 5 to 50 wt% of a release-modifying excipient; and
wherein the matrix is compressible into a pellet.

2. The formulation of claim 1, wherein the phytocannabinoid comprises cannabidiol.

3. The formulation of claim 1, wherein the structural excipient is selected from the group consisting of polycaprolactone, poly(lactic-co-glycolic acid), polylactic acid, polyvinylpyrrolidone, and mixtures thereof.

4. The formulation of claim 1, wherein the release-modifying excipient is selected from the group consisting of comprises stearic acid, magnesium stearate, and mixtures thereof.

5. The formulation of claim 2, wherein the cannabidiol is plant-derived or synthetically produced.

6. The formulation of claim 3, wherein the structural excipient provides structural integrity to the formulation enabling tablebility.

7. The formulation of claim 4, wherein the release-modifying excipient is configured to bind to the phytocannabinoid and control its release from the matrix.

8. A solid matrix comprising the formulation of claim 1.

9. The solid matrix of claim 8, wherein the solid matrix comprises a compressed pellet.

10. The solid matrix of claim 9, wherein the pellet is a 100 mg pellet having of length of 9 mm and a width of 3 mm.

11. The solid matrix of claim 8, further comprising a plurality of pellets each having the formulation of claim 1.

12. A method for preparing a pellet comprising:
compressing a matrix of constituents into the pellet, the matrix of constituents comprising
from 5 to 80 w t% of a phytocannabinoid;
from 5 to 80 wt % of a structural excipient; and
from 5 to 50 wt % of a release-modifying excipient.

13. The method of claim 12, wherein
the phytocannabinoid comprises cannabidiol;
the structural excipient is selected from the group consisting of polycaprolactone, poly(lactic-co-glycolic acid), polylactic acid, polyvinylpyrrolidone, and mixtures thereof;
the release-modifying excipient is selected from the group consisting of comprises stearic acid, magnesium stearate, and mixtures thereof.

14. The method of claim 13, wherein the compressing step comprises
providing a homogenous mixture of the matrix of constituents; and
compressing the homogeneous mixture into a cylindrical pellet.

15. The method of claim 14, wherein the matrix of constituents are mixed by double-cone blender, ribbon blender, or V-blender.

16. The method of claim 15, wherein the homogenous mixture is compressed by stamping press rotary or rotary press.

17. The method of claim 16, wherein the pellet is a 100 mg pellet having of length of 9 mm and a width of 3 mm.
